(19) United States
(12) Patent Application Publication
(10) Pub. No.: US 2005/0070602 A1
(43) Pub. Date: Mar. 31, 2005

(54) Method of modulating immune response by administration of immuno-activation agent

(76) Inventors: Jen-Wei Chiao, Ardsley, NY (US); Longgui Wang, Flushing, NY (US)

Correspondence Address: WINSTON & STRAWN;PATENT DEPARTMENT, 1400 L STREET, N.W., WASHINGTON, DC, 20005-3502, US

(21) Appl. No.: 10/673,426

(22) Filed: Sep. 30, 2003

(51) Int. Cl. A61K 31/26

(52) U.S. Cl. 514/514

(57) ABSTRACT

The present invention is directed to a method of activating or augmenting an immune system of a mammal. The method comprises the step of administering a isothiocyanate (ITC) based agent, such as, N-acetylcysteine conjugate of phenethyl isothiocyanate (PEITC-NAC) or phenethyl isothiocyanate (PEITC), to a mammal in an amount sufficient to activate or augment the immune response in the mammal. The invention is further directed to a method of activating an antigen-specific immune response by administering to a subject an ITC based agent to in an amount sufficient to activate or augment the response of a B cell system or innate immunity to an antigen.

### FIELD OF THE INVENTION

This invention relates generally to methods and compositions of activating or augmenting the immune system and, more particularly, to methods of activating or augmenting an immune response by administering an effective amount of an isothiocyanate (ITC) based agent to a mammal in an amount sufficient to activate or augment the immune responses.

### BACKGROUND ART

The immune system is a body wide defense network of cells and organs that has evolved to defend the body against attacks by “foreign” invaders. Judged by the cell number, distribution, and organs that involved the immune systems are one of the biggest body systems. Lymphocytes are the major cell type of the immune systems. They are generally divided into T and B cell systems, each responsible for the cell-mediated and the humoral immunity. They give rise from multipotential stem cells from the bone marrow, and develop to functional cells through the differentiation and maturation processes. The B cells mature to plasma cells for antibody production. The T cells have many subpopulations with specialized functions. T and B cell system work together with other specialized cells and body systems engaged in constant warfare with microbes that include intracellular and extracellular organisms.

There is also the natural killer (NK) cells of the innate immunity. They are the cells that are spontaneously cytolytic for certain, but by no means all, tumor lines in culture. Their use in the therapy of cancers has been proposed. Modifiers that are able to activate NK cell system may result in increased activity for preventing the initiation, and/or eliminating tumor cells.

One remarkable characteristic of the immune system is the ability to distinguish between self and non-self. Every single cell within the body carries distinctive molecules that mark it as “self”. The normal immune defenses do not attack tissues or cells that carry a self-marker; rather, immune cells coexist peaceably with them in a state known as self-tolerance. The non-self molecules are destroyed by the immune systems.

The proper targets of the immune defenses include infectious organisms—bacteria, fungi, parasites, and viruses, the “foreign” invaders. However, cancer cells are also targets of the immune systems. When normal cells become cancerous, some of the cell's antigens change. These new or altered antigens flag immune defenders, including cytotoxic T cells, natural killer (NK) cells, and macrophages. According to one theory, “patrolling cells” of the immune system provide continuing body wide surveillance, ferret out and eliminating cells that undergoing malignant transformation. Tumors may develop when the surveillance breaks down or is being overwhelmed.

An immune response to a foreign antigen is sometimes characterized by the production of antibodies and the destruction by T lymphocyte of any cells displaying those antigens. Having the ability to activate or augment the immune responses would assist in efforts to eliminate the antigens, and thereby provide a valuable tool to treat and prevent diseases.

It is, therefore, highly desirable to identify relatively inexpensive, non-toxic, easily administered agents which are suitable for enhancing the immune response of mammals. These antigens could be used for accelerating and enhancing the immune responses to prevent and treat diseases and immunodeficiency.

While studying the effects of N-acetylcysteine conjugate of phenethyl isothiocyanate (PEITC-NAC) in vivo, Applicants surprisingly found that when a sufficient quantity of an ITC based agent was administered to a mammal, the ITC based agent activated and/or augmented the immune responses.

PEITC-NAC is a major metabolite of phenethyl isothiocyanate (PEITC), which is a constituent of vegetables of the family of cruciferae (1, 2). PEITC-NAC and several other thiol conjugates of isothiocyanates have been reported as potent cancer chemopreventive agents in a number of experimental animal models (3-7). They induce cytoprotection against carcinogenesis by blocking phase 1 enzymes such as cytochrome P450s that metabolize procarcinogens to carcinogens, and by inducing phase 2 enzymes such as glutathione-S-transferases that remove the eletrophilic metabolites generated from carcinogen metabolism (6, 8). Sulforaphane (SFN), is the predominant isothiocyanate (ITC) found in broccoli, which has been studied for its chemopreventive potential due to its activity in the induction of phase II enzymes involved in carcinogen detoxification and elimination (6). Several laboratory animal studies have shown that phenethyl ITC (PEITC), a principal constituent in watercress, is a potent chemopreventive agent for cancers of the breast, lung, and esophagus (9-11). Recently we have reported that PEITC-NAC significantly inhibited the growth of prostate cancer cells in cultures, with parallel induction of the inhibitors of cyclin-dependent kinases for G₁ arrest (12).

U.S. Pat. No. 6,433,011 is directed to use of PEITC and SFN to inhibit colonic aberrant crypt foci, showing that, based on an animal bioassay with tumors (aberrant crypt foci) initiated by a chemical carcinogen azoxymethane that is known to initiate colon cancer. The preventive mechanism is primarily by inhibiting the enzymes (phase 1) that process the carcinogens, and also by inducing the enzymes (phase 2) that helping to remove the carcinogens via excretion.

U.S. Pat. No. 5,231,209 is also directed to the use of ITC based compounds to prevent and inhibit tumors specially, lung tumors.

By contrast, as stated above, Applicants have now discovered and taught for the first time, that when administered in sufficient quantities ITC based agents are useful in activating and/or augmenting the immune system in a mammal.

### SUMMARY OF THE INVENTION

The present invention is directed to a method of activating or augmenting an immune system of a mammal. The method typically compises administering an isothiocyanate (ITC) based agent to a mammal in need of such treatment an amount effective to activate or augment an immune response in the mammal.

Examples of ITC based agents include: phenyl isothiocyanate (PITC), benzyl isothiocyanate (BITC), phenethyl isothiocyanate (PEITC), N-acetylcysteine conjugate of phenethyl isothiocyanate (PEITC-NAC); 3-phenylpropyl isothiocyanate (PPITC), 4-phenylbutyl isothiocyanate (PBITC), 4-oxo-4-(3-pyridyl)butyl isothiocyanate (OPBITC), 3-phenylpropyl isothiocyanate (PPITC), 4-phenylbutyl isothiocyanate (PBITC); 4-oxo-4-(3-pyridyl)butyl isothiocyanate (OPBITC); and SFN—sulforaphane. In a preferred embodiment, the ITC based agent PEITC-NAC or PEITC.

Preferably the amount of ITC based agent administered is an amount sufficient to activate the B cell system augmenting the production of antibodies specific to an antigen and/or in an amount sufficient to activate the innate immunity by activating a NK cell system. It is often preferable that the treatment results in both an increase in both B and NK cell numbers in the subject being treated. In another embodiment the production of specific antibodies to a specific xenogenic antigen, such as, a specific cancer cell antigen or xenogenic antigen, is activated and/or increased.

Advantageously, the ITC based agent can be administered to a patient having an infection, such as a viral infection like AIDS, SARS, a bacterial infection, or diseases relating to insufficiency in B cell antibody production and NK cell functions.

Typically the agent is administered orally, transdermally, intravenously, or topically, but can also be administered by other means such as, rectally, vaginally, or transmucosally. In one embodiment, the agent is administered systemically in a dietary composition or as a dietary supplement. The compound can advantageously be administered to the mammal in a purified form either alone or in a composition with a pharmacologically acceptable carrier, diluent or with a beverage or foodstuff.

In another embodiment the ITC based agent is administered in combination with a vaccine to augment the immune responses to a particular antigen or antigens, such as one associated with a microbe or autologous cancer cell.

In yet another embodiment of the invention, the agent is administered to a patient having cancer and the amount administered is sufficient to activate the B cell system, augmenting the production of specific antibodies to the cancer cell antigens. In this embodiment it is preferable that the agent be administered in an amount sufficient to also activate the NK cells for anti-tumor activities.

Advantageously, the ITC based agent used usually has low toxicity and can be administered in combination with other agents. The other agents can be selected from agents known to be useful in the particular treatment. Examples of such agents include: radiotherapeutic agents, hormonal therapy agents, immunotherapeutic agents, chemotherapeutic agents, cryotherapeutic agents and gene therapy agents.

The present invention is also directed to a method of activating or increasing the levels of an inhibitor of cyclin-dependent kinase in a mammal. This method comprises: administering an isothiocyanate (ITC) based agent to a mammal in an amount sufficient to increase the levels of an inhibitor of cyclin-dependent kinase. Preferably the inhibitor activated inhibits cyclin-dependent kinase p21^{WAF-1/Cip-1} or p27^{Kip1} and/or the expression of cyclin D and E is reduced. 24. Non-limiting examples of such inhibitors include p151NK4B, p161NK4A, p181NK4C, p191NK4D, p21WAF-1/Cip-1, p27Kip1 and/or p57.

In yet another embodiment of the present invention, the ITC based agent is administered to a patient having cancer and the therapeutic amount administered is sufficient to inhibit Rb phosphorylation in cancer cells in vivo.

As used herein, the abbreviations have the following meanings: BITC—benzyl isothiocyanate; ITCs—isothiocyanates; PBITC—4-phenylbutyl isothiocyanate; PEITC—phenethyl isothiocyanate; PEITC-NAC—N-acetylcysteine conjugate of phenethyl isothiocyanate; PITC—phenyl isothiocyanate; PPITC—3-phenylpropyl isothiocyanate; OPBITC—4-oxo-4-(3-pyridyl)butyl isothiocyanate; SFN—sulforaphane; NAC—N-acetylcysteine. All references cited herein are hereby specifically incorporated into this disclosure by reference.

### BRIEF DESCRIPTION OF THE DRAWINGS

FIGS. 1A-D show activation of immune response of B and NK cells xenografted tumor cells and inhibition of tumor growth by the diet PEITC-NAC. Tumors from all mice were obtained at autopsy after nine weeks of feeding; frozen and paraffin embedded 4-micrometer sections prepared for staining. Representative H & E stained histological sections (×40 objective) shown in FIG. 1A, tumor from mice on control basal diet, and FIG. 1B, tumor from mice on 8 μmol/g PEITC-NAC diet. A significant reduction in mitotic cells (examples indicated by arrow) and the presence of aggregated lymphocytes surrounding the tumor nodels (arrow) were evident in the PEITC-NAC diet group. FIG. 1C, representative tumor section from PEITC-NAC diet group showing lymphocytes immunoperoxidase-stained with antibody recognizing the B220 marker of B cells (arrow). FIG. 1D, tumor section from PEITC-NAC diet group showing cells around tumor nodule immunoperoxidase-stained with CD90.2 indicating NK cells (arrow).

FIG. 2A shows higher antisera titers from mice on the experimental diet against the PC-3 cell antigens. Cultured PC-3 cells were incubated with sera from mice after injected with PC-3 cells as antigen for 6 weeks. Antisera from mice receiving the experimental PEITC-NAC diet (solid lines) show a higher titer than that from mice receiving the basal control diet (dashed lines). An indirect immunofluorescent antibody technique was used for detecting the antibodies with a flow cytometric method.

FIG. 2B shows an increased mean fluorescence intensity on positive cells stained by the antisera from mice with the experimental diet (solid lines) as compared with sera from mice on the control diet (dashed lines). The intensity channels on a flow cytometer analyzed the mean florescence. Each symbol on the lines in the graph indicates the results from three individual mouse+SD.

FIG. 3 shows detection of immunoglobulins in a xenrografted tumor (PC-3) tissue from mice on PEITC-NAC diet by immunostaining with a rat against mouse Ig, wherein the arrows indicate the positive staining, demonstrating the existence of the Ig antibodies within tumor tissue.

FIGS. 4A-B show the induction of apoptosis by PEITC-NAC in the xenografted tumors (PC-3) titue from mice on PEITC-NAC diet by immunostaining with a rat against mouse Ig; wherein FIG. 4A shows increase of apoptotic cells in the tumors from mice fed for nine weeks with PEITC-NAC supplemented diet (▪) as compared to that of mice on control basal diet (□). The apoptotic cells were determined in situ by the presence of DNA strand breaks with TUNEL assay. Vertical bars represent means±SD of eight mice in each diet group, and Student's t test used for comparison of the two groups. FIG. 4B shows western blots performed with pooled total proteins from tumor tissues of each diet group obtained at autopsy. Antibodies used were for total PARP (poly (ADP-ribose) polymerase), 89-kDa fragment of PARP, and β-actin as a loading control.

FIGS. 5A-C show evidences of the dietary effects of PEITC-NAC on proliferation of xenografted tumor cells. In FIG. 5A, mice were injected with BrdU for labeling of proliferating cells that were identified in tumors obtained at autopsy by immunohistochemical staining as described in Materials and Methods below. Vertical bars represent means±SD of proliferating cells of tumors from mice (8 in each diet group) on PEITC-NAC diet (▪) or on control basal diet (□). FIG. 5B, shows western blots performed with pooled total proteins from tumor tissues of each diet group obtained at autopsy. Antibodies against p21, p27, cyclin D1 (cross reactive with D2 and D3), cyclin E, Rb related proteins, or β-actin (loading control) were used. FIG. 5C, graph shows concentration-related reduction of S- and G₂M-phases of PC-3 cells after exposure to PEITC-NAC for 24 hours in cultures (▪). The proportion of G1 cells are indicated by (□). The percentages of cell cycle phases were the means and SD of three separate experiments, estimated from DNA content frequency histograms with a flow cytometric method.

FIGS. 6A-D show that PEITC-NAC diet inhibited cancer growth of xenografted tumors of PC-3 prostate cancer cells in immunodeficient mice. In FIG. 6Am the volumes of xenografted tumors were measured once a week. Basal diet AIN 76A supplemented with PEITC-NAC (8 μmol/g of basal diet) was started with nine mice one week before PC-3 cell injection, and (□) indicates the mean volumes of tumors during the study period. (▪) indicates the mean volumes of control tumors on nine mice fed with basal diet AIN 76A without PEITC-NAC. The numbers above vertical bars are P values comparing the tumor volumes between PEITC-NAC and control diets. FIG. 6B: shows the mean weights of tumors at autopsy; after feeding mice for nine weeks with basal diet (▪) or basal diet supplemented with PEITC-NAC (□). FIG. 6C shows the mean body weights of mice on PEITC-NAC diet (□) or control basal diet (▪) at autopsy. FIG. 6D shows the mean weights of kidneys and spleens from mice on PEITC-NAC (□) or basal diet (▪) at autopsy. Vertical bars in figures are means±SD and Student's t test used for comparison of two groups.

### DETAILED DESCRIPTION OF PREFERRED EMBODIMENT

The present invention is directed to a method of inducing the activation of certain immune systems and enhancing or augmenting the immune responses to an antigen. The invention is based on the Inventors' discovery that ITC based agents, when administered to a mammal in sufficient quantities, activate and/or augment the immune system. Applicants provide and teach herein, a method to activate and increase certain systemic immune responses in a mammal in need of such treatments. The present method typically comprises administering to the mammal a therapeutically effective amount of an ITC based agent. The immune responses activated or augmented by the method typically include the B-cell humoral immunity to produce specific antibodies against antigens and the natural killer cell system (NK cells) against tumor cells.

The term “ITC based agent” refers to compounds wherein isothiocyanate is the base of the compound. Examples of suitable agents includes: phenyl isothiocyanate (PITC), benzyl isothiocyanate (BITC), phenethyl isothiocyanate (PEITC), N-acetylcysteine conjugate of phenethyl isothiocyanate (PEITC-NAC); 3-phenylpropyl isothiocyanate (PPITC), 4-phenylbutyl isothiocyanate (PBITC), 4-oxo-4-(3-pyridyl)butyl isothiocyanate (OPBITC), 3-phenylpropyl isothiocyanate (PPITC), 4-phenylbutyl isothiocyanate (PBITC); 4-oxo-4-(3-pyridyl)butyl isothiocyanate (OPBITC); and SFN—sulforaphane. Examples of preferred ITC based agents include PEITC-NAC or PEITC. One or more ITC based agents may be administered to the mammal.

One skilled in the art may isolate from an appropriate plant, synthesize or buy suitable ITC based agents. For example SFN, PITC, BITC, and PEITC can be obtained from Aldrich Chemical Co. (Milwaukee, Wis.); PPITC can be obtained from Fairfield Chemical Co. (Blythewood, S.C.); and PBITC and OPBITC can be synthesized according to the method disclosed in U.S. Pat. No. 5,231,209. In addition, SFN can be isolated from vegetables, like broccoli and PEITC can be isolated from watercress using techniques such as those disclosed in U.S. Pat. No. 6,348,220, and PEITC-NAC can be synthesized can be prepared by a published method (4, 13) or a modification of a published method (14) as shown in the Example below.

The ITC based agent is preferably administered to the mammal as a purified compound, either alone or in a composition with an acceptable carrier, excipient or diluent or with a beverage or foodstuff. The present agent may be administered in a variety of ways, including topical, enteral, and parenteral routes of administration. For example, suitable modes of administration include subcutaneous, transdermal, transmucosal, including iontophoretic, intravenous, subcutaneous, transnasal, intrapulmonary, transdermal, oral, rectal, vaginal, implantable and the like, as well as their combinations. The particular acceptable form of the therapeutic agent employed will depend on the route of administration selected. A preferred mode of administration is orally, such as, in the form of a dietary supplement, capsule, tablet, syrup, etc. The agent is preferably administered, for example, in a form that enhances its bioavailability when compared with standard oral formulations. Suitable forms include a carrier system that promotes the absorption of compounds through the intestinal epithelium. Examples of these systems are oil-in-water, and water-in-oil emulsions. Exemplary oils that are contemplated for use in oil-in-water and water-in-oil based systems include castor oil, olive oil, soybean oil, safflower oil, coconut oil, cottonseed oil, their combinations, and the like.

Other suitable forms that enhance the bioavailability of the orally administered agent of this invention include single surfactant, and mixed micelle systems. The agent may, for example, be orally administered in the form of a mixed micelle system containing linoleic acid and polyoxyethylene-hardened castor oil. Suitable surfactants contemplated for use in single and mixed micelle systems include polyoxyethylene ether, polyoxypropylene ether, polyoxyethylene lauryl, cetyl and cholesteryl ethers, polyoxyethylene derivatives of lanolin alcohols, and the like, as well as their mixtures.

The agent may be administered as a single dose or in multiple doses. Multiple doses may be administered either continuously, in intervals, or a combination of both. The agent, for example, may be administered as a single dose, optionally coupled with a follow-up dose. The follow-up dose may be administered by the same or different route of administration as a single or sustained dose.

An “effective amount” is defined as an amount of ITC based agent that is capable of producing or increasing an immune response in a subject. In preferred, non-limiting embodiments of the invention, an effective amount of the ITC based agent produces an elevation of the titer of antibacterial or specific antigen antibodies to at least one and half times the antibody titer prior to treatment. In a preferred embodiment the elevation is at least two times the antibody titer prior to treatment with the agent.

A suitable effective amount of the ITC based agent of this invention can be determined by one of skill in the art based upon the level of immune response desired. Such a composition may be administered once, and/or a booster may also be administered. However, suitable dosage adjustments may be made by the attending physician or veterinarian depending upon the age, sex, weight and general health of the human or animal patient.

An immune response may be detected in a mammal by various methods that are well known to those of ordinary skill in the art. Variations in immune response may be detected, for example, by monitoring the levels of antibodies in serum, B and/or T lymphocyte numbers, or their proliferation, or the number of T cell subclasses the helper and/or effector or suppressor T cells.

Similarly, in those embodiments wherein the ITC based agent is mixed with a vaccine, suitable doses of the vaccine composition of the invention can be readily determined by one of skill in the art. The dosage can be adjusted depending upon the human patient or the animal species being treated, i.e. its weight, age, and general health.

An immune response may be detected in a mammal by various methods that are well known to those of ordinary skill in the art. Variations in immune responses may be detected, for example, by monitoring the level of antibodies in serum, B and/or T lymphocyte numbers and proliferation, or T cell subclasses helper and/or effector or suppressor T cells.

As used herein, the “mammal” can be a human or a non-human animal, such as dog, cat, horse, cattle, pig, or sheep for example. Preferably the mammal is human. The term “patient” is used synonymously with the term “mammal” in describing the invention.

Term “augment” and its various grammatical variations refer to the enhancement and/or the increase of immune response in a mammal as compared to the immune response prior to treatment with the ITC based agent.

In a specific embodiment of the invention there is provided a method for treating colon tumor formation in a mammal in need of such treatment. The method comprises administering to the mammal a pharmacologically effective amount of an ITC based agents, such as, PEITC-NAC, SFN and PEITC. PEITC-NAC may undergo reversible dissociation to PEITC and NAC in aqueous solution. Administration of phenethyl isothiocyanate to activate or increase the immune responses in a mammal is also encompassed by the scope of the present invention.

As explained above, the invention is directed to a method of activating or enhancing certain immune systems in a mammal. Advantageously, the method can be used to increase the immune responses against specific antigens. In one embodiment, the ITC agent is administered in combination with an antigen, used in two different routes of administration, for example, one oral and one intradermal, to induce specific immune responses. The ITC based agent can be administered with numerous vaccines, such as, influenza, polo, small pox, etc., to augment the immune response to the vaccine.

The invention is further defined by reference to the following example. It will be apparent to those of ordinary skill in the art that many modifications, both to materials and methods, may be practiced without departing from the purpose and intent of this invention. Thus, the following examples are offered by way of illustration, and not by way of limitation

In the Example below, it was shown that nude mice on a ITC based (PEITC-NAC) diet had consistently smaller tumor volume and weight than the control group. The cellular and molecular responses of the xenografted tumors to PEITC-NAC diet indicated that the growth of the tumor cells was inhibited. The increase of immune cells aggregated around the tumor nodules indicated that a systemic immune response was induced by PEITC-NAC.

These results demonstrated for the first time that the ITC based agents when administered in sufficient quantities activate the immune system and/or increase immune responses.

### EXAMPLE

In the following example, the present inventors demonstrated for the first time that ITC based agents, such as PEITC-NAC, can be used to activate the immune systems and to increase immune responses against the xenogeneic antigens.

In the Example, we examined the biological relevance of PEICT-NAC as, a dietary supplement, on the growth of human prostate cancer cells in vivo as xenografted antigens in immunodeficient mice. We have demonstrated, for the first time, that PEITC-NAC activated the immune systems and enhanced immune responses against the xenogeneic antigens. The immune responses include infiltration of lymphocytes, especially B and NK cells around the tumor nodules, increases of B and NK cell numbers, and the production of more specific antibodies with higher titers against the antigens of the prostate cancer cells.

Materials and Methods

Reagents and Prostate Cancer Cells:

The PEITC-NAC was synthesized by a modification of a published method (14). The product was crystallized from hexane, and purity established by HPLC, NMR and MS; purity was greater than 98%. A human prostate cancer cell line, androgen-independent PC-3 was used as antigen in this invention. PC-3 cells were seeded at 1.5×10⁵ cells/ml in RPMI-1640 containing 15% heat inactivated fetal calf serum with 1% penicillin (10,000 units/ml) and streptomycin (10,000 μg/ml) (15). Cell cycle phase determination was performed using a BD FACScan cytometer according to published procedures (16, 17). The cells were fixed with 80% ethanol at 4° C. and incubated on ice before the DNA was stained with propidium iodide (50 μg/ml).

Xenograft Tumor Assays:

Five week-old BALB/c (nu/nu) male mice purchased from Charles River Laboratories (Wilmington, Mass.) were housed in a barrier facility with 12-h light/dark cycles; tap water and diets provided ad libitum. They were randomly divided into two groups of nine mice. One group provided modified AIN-76A diet (5% corn oil) (basal diet), the other with 8 μmol PEITC-NAC/g of AIN-76A, established as an optimal dose by a separate maximum tolerated dose assay. The diets began one week prior to inoculation of PC-3 cells and continued until termination of experiments. The xenografted tumors were established by a single s.c. injection in the flank of 0.8×10⁶ PC-3 cells suspended in ice-cold matrigel (sigma). The tumor volumes were measured every 7-8 days and calculated by length×width×height×0.5236. Animal body weights were recorded weekly and at autopsy; food consumption was determined twice per week. Two hours before the mice were euthanized, 5-bromo-2′-deoxyuridine (BrdU) (10 mg/kg body weight) was injected in the peritoneum for in vivo labeling of proliferating cells. At autopsy, xenograft tumors were weighed and frozen in liquid nitrogen or fixed in 10% formalin and embedded in paraffin. The BrdU-labeled cells of paraffin embedded tissues on slides were detected employing a monoclonal anti-BrdU antibody from a BrdU detection kit (Roche Molecular Biochemicals) according to manufacturer's direction. The mean numbers of labeled cells from multiple fields were calculated. The comparisons between control and experimental groups were performed with Student's t test.

Analysis of Immune Activation, Apoptosis, and Protein Expression:

After 30 days of administration of PEITC-NAC, the quantity of B cells and NK cells in the peripheral blood of all mice were determined by direct fluorescent antibody technique with the flow cytometric method. Monoclonal antibodies against mouse B cell antigen CD19 or against Pan NK cell antigen CD49b were used. A mouse isotype control was used as a background.

The reaction of serum antibodies from mice on the experimental or control diet with cultured PC-3 cells was analyzed with an indirect immunofluorescent antibody technique. A fluoresecin-labeled goat IgG-(Fab′)₂ antibody against mouse Ig was used as the second antibody. PC-3 cells stained with a nonspecific mouse IgG as the first antibody were used as a background. The proportion of the positively stained cells and the mean fluorescence intensity were analyzed with the intensity channel on a flow cytometer.

The changes of B and NK cells were further examined in the-frozen and paraffin embedded xenografted tumor tissues by for immunohistochemical staining. Mouse B cells were identified with a primary monoclonal rat anti-B220 antibody, and NK cells of immunodeficient mice detected with a rat anti-mouse CD90.2 (18) primary antibody (BD PharMingen). A biotin-conjugated goat anti-rat Ig was used as a secondary antibody, with chromogen color development using an ImmunoCruz Staining System (Santa Cruz Biotechnology). A rat IgG_{2aK} was used as an isotype control. The binding of mouse Ig to xenografted tumor cells was determined by a direct antibody staining method using a biotinylated goat anti-mouse Ig (BD PhaMingen), or an isotype Ig control.

The apoptotic cells of xenografted tumors were determined with paraffin embedded tissue slides by the characteristic nucleus morphology, and by the presence of DNA strand breaks using the terminal deoxynucleotidyl tranferase-mediated biotinylated UTP nick end labeling (TUNEL). An in situ detection kit from Roche Molecular Biochemicals, Indianapolis, Ind. was employed according to manufacturer's direction (17). Multiple fields, but not necrotic tissues, were determined for apoptotic cells.

The protein levels of xenografts were determined by Western blot analyses using standard procedures (19). Total proteins were prepared from each group of pooled individual xenografts of equal weight. The tissues were homogenized in the presence of a lysis buffer with protease inhibitors (17) and lysates collected after centrifugation at 4° C. Polyclonal antibodies against human p21^{WAF-}1/Cip-1, p27^{Kip1}, cyclin E, and an anti-cyclin D1 cross reactive with cyclins D2 and D3, an anti-Rb reactive with both phosphorylated and non-phosphorylated proteins, and an antibody against intact poly (ADP-ribose) polymerase (PARP) and the 89 kDa fragment were purchased from Santa Cruz Biotechnology. Preferably the endogenous cyclin-dependent kinase inhibitors are p151NK4B, p161NK4A, p181NK4C, p191NK4D, p21WAF-1/Cip-1, p27Kip1 and/or p57 that inhibit cyclin-dependent kinases.

Results

Activation of the Immune systems and Enhancement of Immune Responses by PEITC-NAC Diet:

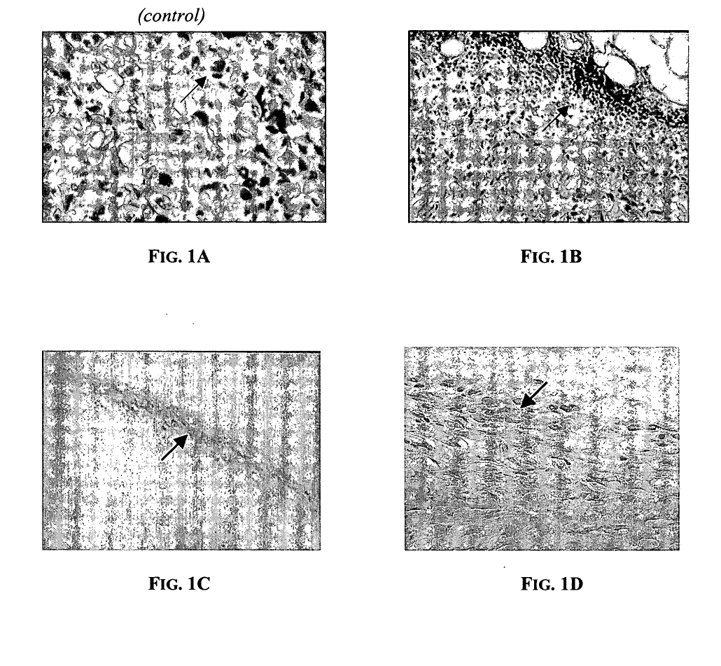

Tissue sections of the xenografted tumors from mice on PEITC-NAC or control diet all had central necrosis surrounded by neoplastic cells that were large, containing a large vesicular nucleus and a prominent nucleolus (FIGS. 1A and 1B). Occasional multinucleated giant cells were present. Numerous, focally abnormal mitosis was evident. Tumors from PEITC-NAC fed mice had less mitotic figures, averaged three per high power field (FIG. 1B), than the control tumors which had greater than five mitotic figures per field (FIG. 1A). Apoptotic cells as evidenced by condensed cytoplasm and pyknotic hyperchomatic nuclei were numerous, especially rimming the area of central necrosis of the control tumors. Apoptotic cells in the experimental tumors were seen rimming the area of central necrosis and also within areas of viable tumors.

Tumor nodules from experimental but not the control mice were surrounded by aggregates of small lymphocytes, shown representatively in FIG. 1B. The great majority of them were B cells bearing marker B220 as identified by immunohistochemical staining (FIG. 1C). Small quantity of lymphocytes stained positive for CD90.2, marker for T and NK cells (18), were also detected infiltrating around the tumor nodules (FIG. 1D). These cells may be NK cells since there are no T cells in immunodificient mice.

The quantities of B cells and NK cells in the peripheral blood of all mice were determined by direct immunofluorescent antibody technique with the flow cytometric method. Monoclonal antibodies against mouse B cell antigen CD19 or against mouse Pan NK cell antigen CD49b were used. Table 1 shows that 30 days after the xenografting of human prostate cancer PC-3 cells the numbers of B and NK cells were increased in the peripheral blood of the experimental mice receiving PEITC-NAC diet, as compared to that of mice on control diet without PEITC-NAC. This has indicated that PEITC-NAC activated the immune systems in recognition of the presence of antigens of PC-3 cells.

TABLE 1
Increase of B and NK cells in the peripheral blood of mice on
PEITC-NAC diet*.

| Group | Mean of B Cells (%) | Mean of NK Cells (%) |
|---|---|---|
| Control mice 1 | 26.1 | 21.9 |
| Control mice 2 | 35.0 | 27.0 |
| Average of Control | 30.5 | 24.4 |
| Experimental mice 1 | 38.3 | 26.0 |
| Experimental mice 2 | 45.8 | 27.6 |
| Experimental mice 3 | 42.1 | 38.0 |
| Average of experimental | 42.0 | 30.5 |
| mice | | |

*The results are expressed as a percentage of the total monocnuclear cells (10,000) that were collected for analyses. All nude mice were xenografted with androgen-insensitive human prostate cancer PC-3 cells as described in the “Materials and Methods”. The experimental mice received PEITC-NAC diet (8 μmol/g of AIN-76A) while the control mice received basal diet AIN-76A without PEITC-NAC.

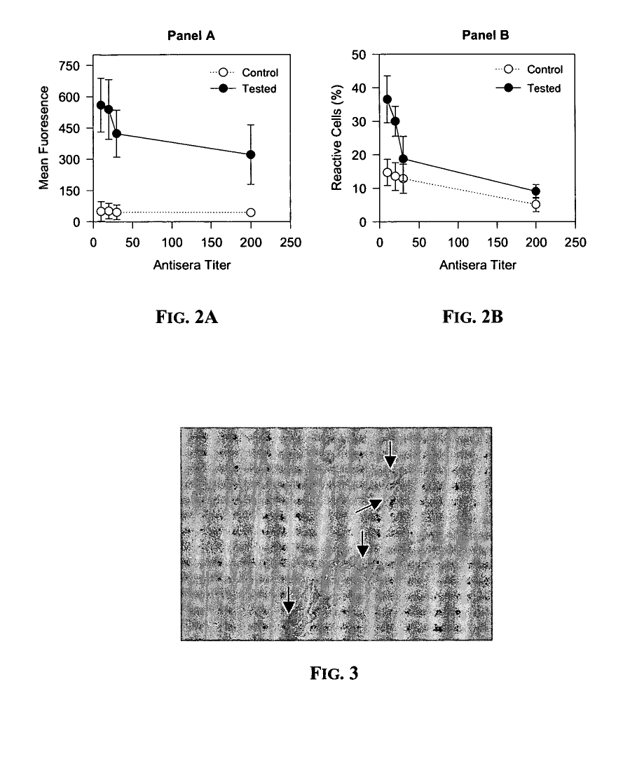

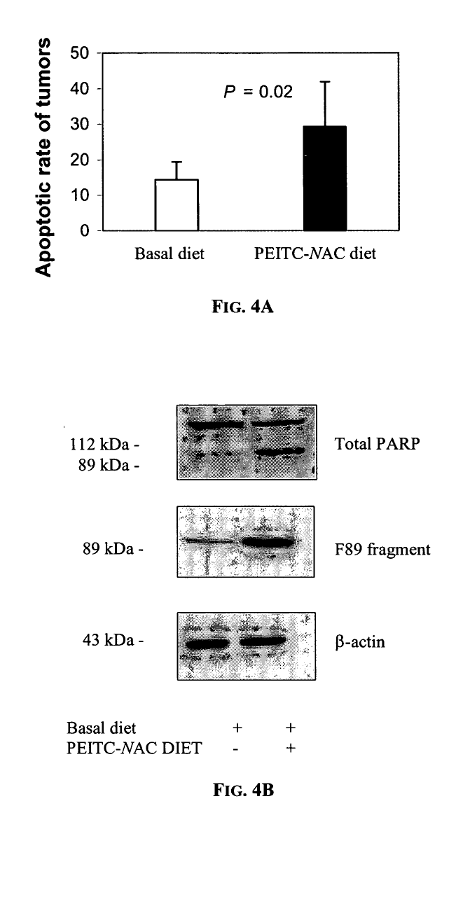

Mouse sera were incubated with cultured PC-3 cells to detect the presence and reactivity of antibodies against PC-3 antigens using an indirect immunofluorescent antibody technique. The proportion of the positively stained cells and the mean fluorescence intensity were analyzed by a flow cytometric method. FIG. 2A shows that significantly more PC-3 cells were detected with the antisera from mice treated with the experimental diet than the antisera from mice on control diet, based on the same serum titers. For example, at the titer of 20, the antisera from mice on experimental diet detected approximately 24-38% cells while approximately 7-16% cells detected with the sera from mice on the control diet. The data indicate that the experimental diet resulted in the production of antibodies with higher titer against the PC-3 cells, which were used as the antigen. Additionally, the mean fluorescence on the stained PC-3 cells was significantly more intense with the antisera from mice using the experimental diet, as compared to the control mice sera (FIG. 2B). The results indicate clearly that there were more antibodies bound with the antigens on PC-3 cells, implying the presence of more specific antibodies after the diet treatment.

To determine the presence of mouse antibodies against the xenografted PC-3 tumor cells, tissue sections from the experimental and control tumors were stained with immunoperoxidase procedures using a specific antibody against mouse immunoglobulins, or with a control isotype antibody. Compared to the control antibody, only cells from the experimental but not the control tumors were demonstrated to have bound mouse immunoglobulins (FIG. 3). This indicates that PEITC-NAC activated the B cell system and enhanced the antibody production against the antigens of human PC-3 tumor cells (FIG. 3).

A complex receptor on NK cells, NKG2A/C/E was also demonstrated among cells infiltrating around the tumor nodules from PEITC-NAC treated mice with immunohistochemical staining. They are part of the complex receptor of NK cells mediating signal transduction for NK cell activities. Their demonstration indicates the presence of functional NK cells.

Increase in Apoptotic Rate:

The apoptotic cells with DNA strand breaks in xenografted tumors was determined in situ by TUNEL assay. The average apoptotic rate of experimental tumors elevated approximately two folds, from 14.4% to 29.29% (P=0.02) as compared to control tumors (FIG. 4A). Apoptosis was further confirmed by the cleavage of PARP, target of proteolysis of caspases that execute apoptosis with Western blot analyses. Significant increase of the cleaved fragments, including a signature 89 kDa apoptotic fragment was detected in the experimental tumors (FIG. 4B).

Inhibition of Cell Cycle Progression:

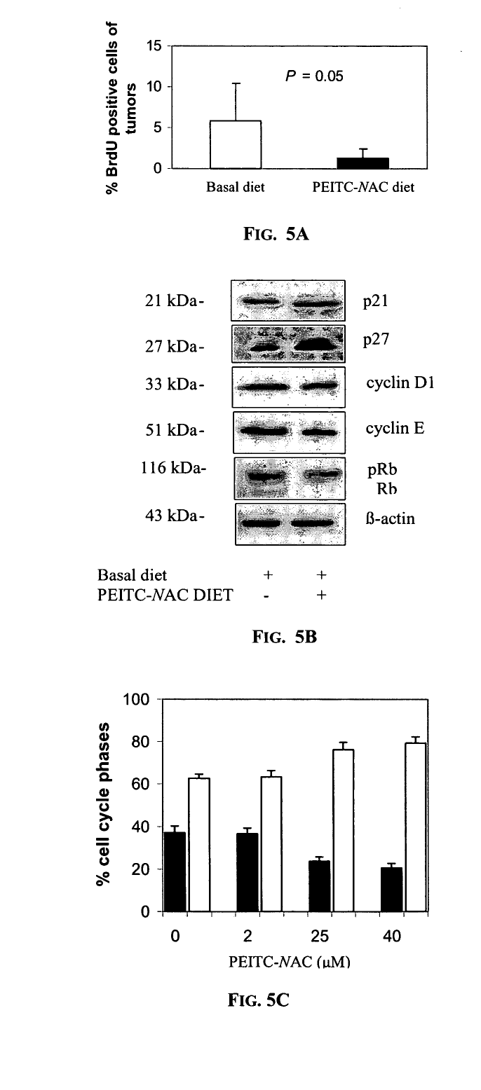

All mice were injected with BrdU for labeling proliferating cells and the results of immunohistochemical staining of the xenografted tumors presented in FIG. 5A. The proliferating cells were approximately 75% less in tumors from experimental mice versus control mice, indicating reduced DNA synthesis. Levels of inhibitors of cyclin-dependent kinases (cdk) p21 and p27 that affect cell cycle progression were examined with the xenografted tumors by Western blot analyses. An increased p21 and p27 levels (32% and 40%), along with a reduced expression of cyclins D1 (32%) and E (42%) were determined in the pooled experimental tumors as compared to the controls (FIG. 5B). The PEITC-NAC effects were further examined on phosphorylation of Rb, because an induction of cdk inhibitors or decrease in cyclins could lead to decreased cdk activity that affects down-stream phosphorylation of Rb proteins as regulators of G₁- to S-phase transition (20, 21, 22). FIG. 5B shows a significant reduction of Rb phosphorylation in experimental tumors as compared to controls. Equal protein loading was confirmed by probing the Western blots with an anti-β actin antibody (FIG. 5B).

PEITC-NAC Diet Inhibited Tumor Growth of Xenografts:

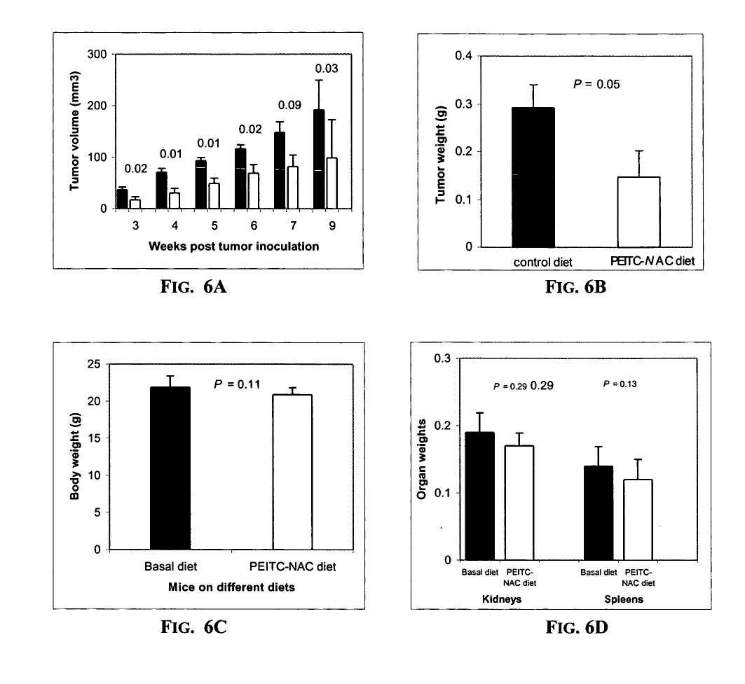

Dietary effects of PEITC-NAC on the growth of xenografted tumors of human prostate cancer PC-3 cells in immunodeficient mice were evaluated. Mice on control basal diet without PEITC-NAC developed palpable tumors 7-10 days after inoculation of PC-3 cells, with 100% tumor incidence. The tumors in mice with experimental PEITC-NAC diet took longer a time period, 7-22 days to be palpable, with 88% tumor incidence. FIG. 6A shows that the PEITC-NAC diets suppressed the tumor growth. The tumor volumes were smaller soon after the tumors became palpable, and persisted during the subsequent study period as compared to control tumors (P<0.05). Tumor inhibition was noted in 100% of mice fed with PEITC-NAC diet. Both smaller and larger (>100 mm³) tumors from the early and late study period were inhibited. The weight of tumors determined at autopsy confirmed that the tumors after feeding with PEITC-NAC diet were approximately 50.2% smaller (P=0.05) (FIG. 6B).

Animals were observed throughout the study period and showed no signs of unusual behavior with the PEITC-NAC diet. At termination, the average body weight of experimental mice was 20.93 g compared to control mice 21.89 g, corresponding to approximately 4.4% less in the experimental mice (P=0.11) (FIG. 6C). The average weights of kidneys and spleens from experimental mice were similarly less than the controls, but the difference was not significant (P>0.13) (FIG. 6D) indicating no overt toxicity with the PEITC-NAC diet. Measurement of food consumption revealed that mice on PEITC-NAC diets ate approximately 6.5% less daily as compared to control mice, perhaps due to palatability.

Discussion

The Inventors have surprisingly discovered and now shown that ITC based agents (PEITC-NAC) induce activation and enhancement of the immune responses. In the example the ITC based agent inhibited the growth of the tumors by activating and/or augmenting the immune response to the xenogeneic antigens. Nude mice on the PEITC-NAC diet had consistent smaller tumor volume and weight throughout the study period. The cellular and molecular responses of the xenografted tumors to PEITC-NAC diet indicated that the growth of the tumor cells was inhibited. The increase of immune cells aggregated around the tumor nodules indicates further that a systemic immune response was induced by PEITC-NAC.

The mice on the PEITC-NAC diet weighed marginally less, approximately 4%, than mice on control diets without PEITC-NAC. The extent of the body weight disparity is probably too small to account for a 50% reduction in the tumor weight. The differences of body weight were likely caused by a reduced intake of the food with PEITC-NAC, as supported by the food consumption data. The reduced food intake could be due to palatability, observed also previously that A/J mice ate less food supplemented with PEITC-NAC but it was not a factor to change the size of carcinogen-induced lung tumors (5).

Lymphocyte infiltration around the peripheral of the experimental tumor nodules revealed an activation of the immune systems. The detection of B cells with marker B220 among the infiltrated lymphocytes, the increases of B cell number in the peripheral blood of these mice, and the binding of mouse antibodies to tumor cells indicated the activation of the B cell system. The xenografted prostate cancer PC-3 cells served as a source of antigenic signals. In addition cells with CD90.2 were increased around the experimental tumors. NK cells from BALB/c immunodeficient mice were predominantly CD90.2 positive (18) showing that NK cells are also activated. The reactions of the mouse antisera with cultured PC-3 cells further indicated that mice treated with PEITC-NAC diet had more specific antibodies with higher titers than the mice sera with the control diet. These findings provide evidence of isothiocyanate's role in increasing immunity against antigens.

The effects of PEITC-NAC on xenografted PC-3 cells show that PEITC-NAC blocked cell cycle progression by modulating the expression and function of cell cycle regulators. PEITC-NAC induced signals those up-regulate cdk inhibitors p21 and p27, and reduced the expression of cyclins D and E. This would effectively block the G1-phase progression, because the progression is mediated by the combined activity of cyclin D1/cdk4, 6 and cyclin E/cdk 2 (22). As a result, phosphorylated Rb proteins, which activate the transition from G1-to-S (22), was decreased and the cell cycle progression retarded. The presence of apoptotic cells was increased in the same tumors, demonstrating that the cells undergo apoptosis after growth arrest. Whether the growth suppression and apoptosis of the xenograph induced by PEITC-NAC were the results of the immunological reactions against the tumors, are currently being investigated.

Although preferred embodiments of the invention have been described in the foregoing description, it will be understood that the invention is not limited to the specific embodiments disclosed herein but is capable of numerous modifications by one of ordinary skill in the art. It will be understood that the materials used and the chemical details may be slightly different or modified from the descriptions herein without departing from the processes and compositions disclosed and taught by the present invention.

### References

1. Brusewitz, G., Cameron, B. D., Chasseaud, L. F., Gorler, K., Hawkins, D. R., Koch, H., and Mennicke, W. H. (1977) The metabolism of benzyl isothiocyanate and its cysteine conjugate. The Biochemical Journal, 162: 99-107.
2. Eklind, K. I., Morse, M. A., and Chung, F. L. (1990) Distribution and metabolism of the natural anticarcinogen phenethyl isothiocyanate in A/J mice. Carcinogenesis, 11: 2033-2036.
3. Hecht, S. S. (1995) Chemoprevention by isothiocyanates. Journal of Cellular Biochemestry (Suppl.), 22: 195-209.
4. Jiao, D., Conaway, C. C., Wang, M. H., Yang, C. S., Koehl, W., and Chung, F. L. (1996) Inhibition of N-nitrosodimethylamine demethylase in rat and human liver microsomes by isothiocyanates and their glutathione, L-cysteine, and N-acetyl-L-cysteine conjugates. Chemical Research in Toxicology, 9: 932-938.
5. Yang, Y. M., Conaway, C. C., Chiao, J. W., Wang, C. X., Amin, S., Whysner, J., Dai, W., Reinhardt, J., and Chung, F. L. (2002) Inhibition of benzo(a)pyrene-induced lung tumorigenesis in A/J mice by dietary N-acetylcysteine conjugates of benzyl and phenethyl isothiocyanates during the postinitiation phase is associated with activation of mitogen-activated protein kinases and p53 activity and induction of apoptosis. Cancer Research, 62: 2-7.
6. Zhang, Y. and Talalay, P. (1994) Anticarcinogenic activities of organic isothiocyanates: chemistry and mechanisms. Cancer Research (Suppl.), 54: 1976s-1986s.
7. Chung, F.-L. (1992) “Chemoprevention of lung carcinogenesis by aromatic isothiocyanates.” In. Cancer Chemoprevention, Edition, (eds., Wattenberg, L., Lipkin, M., Boone, C. W., Kelloff, G. J.) pp. 227-245, CRC Press Inc.
8. Yang, C. S., Smith, T. J., and Hong, J. Y. (1994) Cytochrome P-450 enzymes as targets for chemoprevention against chemical carcinogenesis and toxicity: opportunities and limitations. Cancer Research, 54: 1982s-1986s.
9. Wattenberg, L. W. (1977) Inhibition of carcinogenic effects of polycyclic hydrocarbons by benzyl isothiocyanate and related compounds. Journal of the National Cancer Institute, 58: 396-398.
10. Chung, F.-L., Morse, M. A., and Eklind, K. I. (1992) New potential chemopreventive agents for lung carcinogenesis of tobacco-specific nitrosamine. Cancer Research, 52: 2719s-2722s, 1992.
11. Stoner, G. D., Morrissey, D. T., Heur, Y.-H., Daniel, E. M., Galati, A. J., and Wagner, S. A. (1991) Inhibitory effects of phenethyl isothiocyanate on N-nitrosobenzyl-methylamine carcinogenesis in the rat esophagus. Cancer Research, 51: 2063-2068.
12. Chiao, J. W., Chung, F., Krzeminski, J., Amin, S., Arshad, R., Ahmed, T., and Conaway, C. C. (2000) Modulation of growth of human prostate cancer cells by the N-acetylcysteine conjugate of phenethyl isothiocyanate. International Journal of Oncology, 16: 1215-1219.
13. Kassahun, K., Davis, M., Hu, P., Martin, B., and Baillie, T. (1997) Biotransformation of the naturally occurring isothiocyanate sulforaphane in the rat: identification of phase 1 metabolites and glutathione conjugates. Chemical Research in Toxicology, 10: 1228-1233.
14. Chung, F. L., Morse, M. A., Eklind, K. I., and Lewis, J. (1992) Quantitation of human uptake of the anticarcinogen phenethyl isothiocyanate after a watercress meal. Cancer Epidemiology, Biomarkers & Prevention: A Publication of the American Association for Cancer Research, cosponsored by the American Society of Preventive Oncology, 1: 383-388.
15. Wang, L. G., Liu, X. M., Kreis, W., and Budman, D. R (1999) Phosphorylation/dephosphorylation of androgen receptor as a determinant of androgen agonistic or antagonistic activity. Biochemical and Biophysical Research Communications, 259: 21-28.
16. Telford, W. G., King, L. E., and Fraker, P. J. (1992) Comparative evaluation of several DNA binding dyes in the detection of apoptosis-associated chromatin degradation by flow cytometry. Cytometry: the Journal of the Society for Analytical Cytology, 13: 137143.
17. Chiao, J. W., Chung, F. L., Kancherla, R., Ahmed, T., Mittelman, A., and Conaway, C. C. (2002) Sulforaphane and its metabolite mediate growth arrest and apoptosis in human prostate cancer cells. International Journal of Oncology, 20: 631-636.
18. Mattes, M. J., Sharrow, S. O., Herberman, R. B., and Holden, H. T. (1979) Identification and separation of Thy-1 positive mouse spleen cells active in natural cytotoxicity and antibody-dependent cell-mediated cytotoxicity. Journal of Immunology, 123: 2851-2860.
19. Sherr, C. J. (2000) The Pezcoller lecture: cancer cell cycles revisited. Cancer Research, 60: 3689-3695.
20. Hunter, T. and Pines, J. (1994) Cyclins and cancer. II: Cyclin D and CDK inhibitors come of age. Cell, 79: 573-582.
21. Morgan, D. O. (1995) Principles of CDK regulation. Nature, 374: 131-134.
22. Sherr, C. J. and Roberts, J. M. (1999) CDK inhibitors: positive and negative regulators of G1-phase progression. Genes and Development, 13: 1501-1512.

1. A method of activating or augmenting an immune system of a mammal which comprises administering an isothiocyanate (ITC) based agent to a mammal in need of such treatment in an amount effective to activate or augment an immune response in the mammal.

2. The method of claim 1, wherein the ITC-based agent is N-acetylcysteine conjugate of phenethyl isothiocyanate (PEITC-NAC) or phenethyl isothiocyanate (PEITC).

3. The method of claim 1, wherein the ITC-based agent activates or augments the production of specific antibodies of a B cell system to an antigen.

4. The method of claim 1, wherein the agent activates or augments innate immunity.

5. The method of claim 4, wherein the ITC-based agent activates or augments a NK cell system.

6. The method of claim 1, wherein the mammal is a patient having an immunodeficiency or an infection.

7. The method of claim 6, wherein the patient has AIDS or SARS.

8. The method of claim 1, wherein the mammal is a patient having cancer, and the ITC-based agent is administered in an amount sufficient to activate a B cell immune system by production of one or more antibodies specific to a cancer antigen.

9. The method of claim 8, wherein the ITC-based agent activates the NK cell system to destroy cancer cells.

10. The method of claim 8, wherein the ITC-based agent is administered in combination with an additional agent selected from the group consisting of radiotherapeutic agents, hormonal therapy agents, immunotherapeutic agents, chemotherapeutic agents, cryotherapeutic agents and gene therapy agents.

11. The method of claim 1, wherein the ITC-based agent is administered in combination with a vaccine to augment the immune response to the vaccine.

12. The method of claim 1, wherein the mammal is one that is suffering from a condition relating to insufficient T-cell functions and the amount of the ITC-based agent administered augments the immune response of the B cell and innate immunity systems in the mammal.

13. The method of claim 1, wherein the mammal is one that suffers from an infection wherein the amount of the ITC-based agent administered augments the production of antibody specific to the infectious agent.

14. The method of claim 1, wherein the ITC-based agent is administered orally, intravenously, or topically.

15. The method of claim 14, wherein the ITC-based agent is administered systemically in a dietary composition or supplement.

16. A method of activating or augmenting an antigen-specific immune response in a subject in need of such treatment which comprises administering to the subject an isothiocyanate (ITC) based agent in an amount effective to activate or augment a B cell system response or an innate immunity response to an antigen.

17. The method of claim 16, wherein the ITC-based agent is N-acetylcysteine conjugate of phenethyl isothiocyanate (PEITC-NAC) or phenethyl isothiocyanate (PEITC).

18. The method of claim 16, wherein B and NK cell numbers augment.

19. The method of claim 18, wherein the ITC-based agent is administered to activate or augment production of antibodies specific to a cancer cell antigen.

20. The method of claim 17, wherein the PEITC-NAC or PEITC is administered in combination with an additional agent selected from the group consisting of radiotherapeutic agents, hormonal therapy agents, immunotherapeutic agents, chemotherapeutic agents, cryotherapeutic agents and gene therapy agents.

21. The method of claim 16, wherein the antigen is associated with a xenogeneic cell or antigen.

22. The method of claim 16, wherein the ITC-based agent is administered in combination with a vaccine.

23. The method of claim 21, wherein the mammal is a patient that has cancer and the therapeutic amount of an isothiocyanate (ITC) based agent is sufficient to increase levels of endogenous cyclin-dependent kinase inhibitors.

24. The method of claim 23, wherein the endogenous cyclin-dependent kinase inhibitors are p151NK4B, p161NK4A, p181NK4C, p191NK4D, p21WAF-1/Cip-1, p27Kip1 and/or p57.

25. The method of claim 23, wherein the inhibitors inhibit cyclin-dependent kinases.

26. The method of claim 22, wherein the expression of cyclin D and E is reduced.

27. The method of claim 23, wherein the ITC-based agent is N-acetylcysteine conjugate of phenethyl isothiocyanate (PEITC-NAC) or phenethyl isothiocyanate (PEITC).

28. The method of claim 26, wherein the mammal is a patient that has cancer and the therapeutic amount of PEITC-NAC or PEITC is administered to inhibit Rb phosphorylation in cancer cells in vivo.
